(19) United States
(12) Patent Application Publication
(10) Pub. No.: US 2006/0173270 A1
(43) Pub. Date: Aug. 3, 2006

(54) Adaptable user interface for diagnostic imaging

(76) Inventors: Allison L. Weiner, Milwaukee, WI (US); Robert F. Senzig, Germantown, WI (US)

Correspondence Address: PATRICK W. RASCHE (12553 - 1000), ARMSTRONG TEASDALE LLP, ONE METROPOLITAN SQUARE, SUITE 2600, ST. LOUIS, MO, 63102-2740, US

(21) Appl. No.: 11/286,683

(22) Filed: Nov. 23, 2005

Related U.S. Application Data
(60) Provisional application No. 60/630,970, filed on Nov. 24, 2004.

(51) Int. Cl. A61B 5/05

(52) U.S. Cl. 600/407

(57) ABSTRACT

A method of operating a user interface is provided. The method includes receiving a first control command at a console operationally coupled to a first and second medical system, wherein the console has control over both the first and second medical systems. The first medical system is operated based upon the first control command. The method further includes receiving a second control command at the console. The second medical system is operated based upon the second control command.

### CROSS REFERENCE TO RELATED PATENTS

This application claims the benefit of U.S. provisional application No. 60/630,970 filed Nov. 24, 2004, which is herein incorporated in its entirety.

### BACKGROUND OF THE INVENTION

This invention relates generally to medical systems for scanning and analyzing imaging data of patients. As medical imaging technology advances, the skills required of an operator become increasingly demanding. Scanning is very fast in modern scanners, making image acquisition and analysis more interactive. Scanning may also be conducted by an operator using a number of imaging modality systems.

During planning and diagnosis of a medical imaging procedure, the imaging system does not provide patient history, genetic makeup, and other relevant patient information to the radiologist. Nor does an imaging system provide an automatic analysis and comparison of patients with similar history and a statistical projection of likelihood of proper diagnosis from the medical imaging system to the radiologist, to assist with diagnosis during and immediately following the imaging procedure.

Accordingly, there is a need for a user interface that is adaptable to the needs of its operators, and adaptable to different modes of operation and with different imaging modalities, such that the interface is recognizable from one modality to the next, and from one console to the next. There is also a need for an imaging system to automatically analyze data acquired from a medical imaging system and color code the results to provide a statistically-based interpretation of results against a database.

### BRIEF DESCRIPTION OF THE INVENTION

In one aspect, a method of operating a user interface is provided. The method includes receiving a first control command at a console operationally coupled to a first and second medical system, wherein the console has control over both the first and second medical systems. The first medical system is operated based upon the first control command. The method further includes receiving a second control command at the console. The second medical system is operated based upon the second control command.

In another aspect, Another method of operating a user interface is provided. The method includes receiving a first control command at the user interface. The first control command pertains to a first scan of an object using a first imaging device. The first control command is performed by the first imaging device. The method further includes receiving a second control command at the user interface, wherein the second control command pertains to a second scan of an object using a second imaging device. The second imaging device performs the second control command.

In a further aspect, another method of operating a user interface is provided. The method includes receiving predetermined thresholds for imaging data and receiving a set of imaging data of an object. The method further includes comparing the imaging data and the thresholds. A characteristic of the object is automatically determined based upon the predetermined thresholds and the imaging data.

In another aspect, a user interface is provided. The user interface includes at least two medical systems operatively coupled to a control console. The control console has simultaneous control over both of the two medical systems.

In a further aspect, a computer program embodied on a computer readable medium for operating a user interface is provided. The computer program includes at least one code segment that receives a first control command at a console operationally coupled to a first and second medical system, wherein the console has control over both medical systems. The first medical system is operated in accordance with the first control command. The computer program further includes at least one code segment that receives a second control command at the console and operates the second medical system in accordance with the second control command.

### BRIEF DESCRIPTION OF THE DRAWINGS

FIG. 1 is a dual modality imaging system for scanning a patient.

FIG. 2 illustrates a CT system, as one of a plurality of imaging systems that may be used in a multi-modality imaging system, with a user interface.

FIG. 3 illustrates an example of, but not limited to, four primary icons, console, viewport, communication center, and monitor, which may be configured using a state changer.

FIG. 4 illustrates examples of icons that a state changer may exhibit, for instance scanning command, stop command, security access, or a switch to analysis mode.

FIG. 5 illustrates examples of console displays.

FIG. 6 illustrates examples of viewport options.

FIG. 7 illustrates a communication center.

FIG. 8 illustrates an example of a communication center.

FIG. 9 illustrates examples of user interfaces and configurations.

FIG. 10 illustrates a plurality of systems operable by any and all of a plurality of consoles.

### DETAILED DESCRIPTION OF THE INVENTION

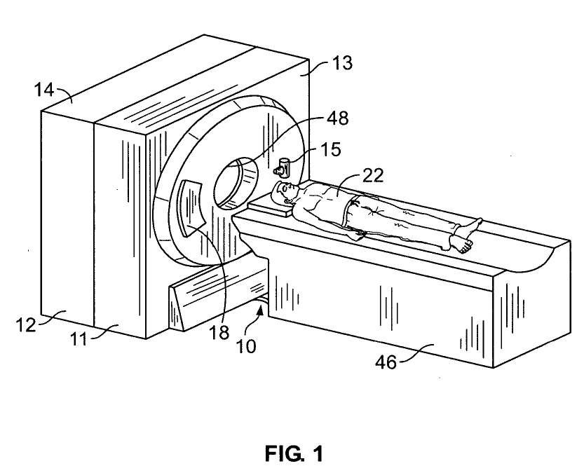

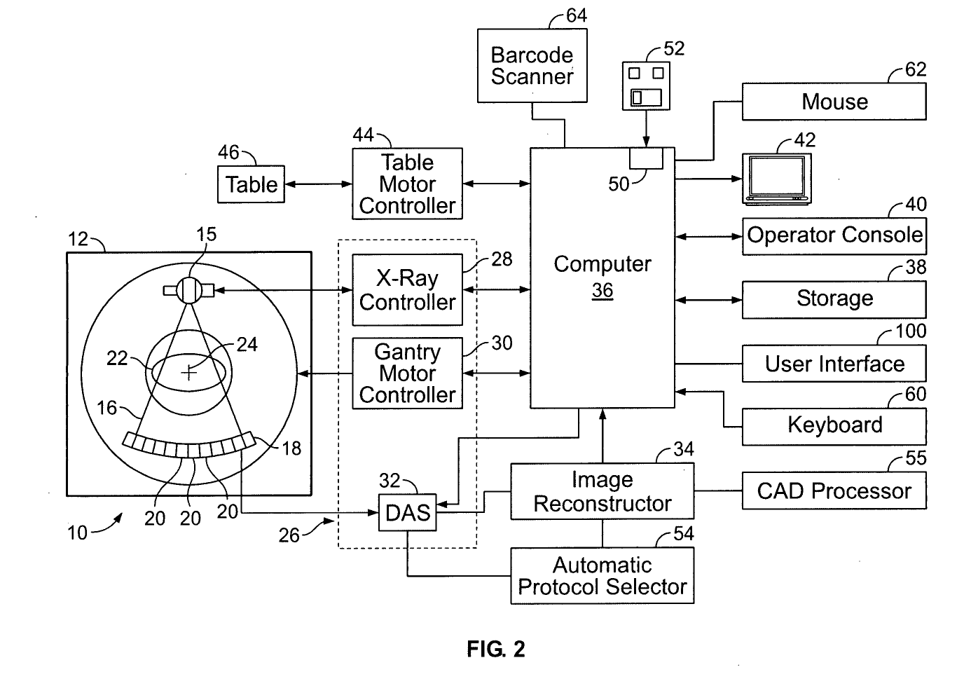

FIG. 1 is a perspective view of an exemplary imaging system 10. FIG. 2 is a schematic block diagram of imaging system 10 (shown in FIG. 1). In the exemplary embodiment, imaging system 10 is a multi-modal imaging system and includes a first modality unit 11 and a second modality unit 12. Modality units 11 and 12 enable system 10 to scan an object, for example, a patient, in a first modality using first modality unit 11 and to scan the object in a second modality using second modality unit 12. System 10 allows for multiple scans in different modalities to facilitate an increased diagnostic capability over single modality systems. In one embodiment, multi-modal imaging system 10 is a Computed Tomography/Positron Emission Tomography (CT/PET) imaging system 10. CT/PET system 10 includes a first gantry 13 associated with first modality unit 11 and a second gantry 14 associated with second modality unit 12. In alternative embodiments, modalities other than CT and PET may be employed with imaging system 10. Gantry 13 includes first modality unit 11 that has an x-ray source 15 that projects a beam of x-rays 16 toward a detector array 18 on the opposite side of gantry 13. Detector array 18 is formed by a plurality of detector rows (not shown) including a plurality of detector elements 20 that together sense the projected x-rays that pass through an object, such as a patient 22. Each detector element 20 produces an electrical signal that represents the intensity of an impinging x-ray beam and therefore, allows estimation of the attenuation of the beam as it passes through object or patient 22.

During a scan, to acquire x-ray projection data, gantry 13 and the components mounted thereon rotate about an examination axis 24. FIG. 2 shows only a single row of detector elements 20 (i.e., a detector row). However, a detector array 18 may be configured as a multislice detector array having a plurality of parallel detector rows of detector elements 20 such that projection data corresponding to a plurality of slices can be acquired simultaneously during a scan. To acquire emission data, gantry 14 rotates one or more gamma cameras (not shown) about examination axis 24. Gantry 14 may be configured for continuous rotation during an imaging scan and/or for intermittent rotation between imaging frames.

Following is a discussion of the operation of a CT scanner. User interface 100 may be used for interfacing with a CT system, PET, MR, or other system. The following discussion is presented as a means to demonstrate a system (CT in this case) and how a user interface may be used to control the system. The rotation of gantries 13 and 14, and the operation of x-ray source 15 are controlled by a control mechanism 26 of CT/PET system 10. Control mechanism 26 includes an x-ray controller 28 that provides power and timing signals to x-ray source 15 and a gantry motor controller 30 that controls the rotational speed and position of gantry 13 and gantry 14. A data acquisition system (DAS) 32 of control mechanism 26 samples data from detector elements 20 and the gamma cameras and conditions the data for subsequent processing. An image reconstructor 34 receives sampled and digitized x-ray data and emission data from DAS 32 and performs high-speed image reconstruction. The reconstructed image is transmitted as an input to a computer 36 which stores the image in a storage device 38.

Computer 36 also receives commands and scanning parameters from an operator via a console 40 that has an input device, such as, a keyboard 60, a mouse 62, or a barcode scanner 64. An associated display 42 allows the operator to observe the reconstructed image and other data from computer 36. The operator supplied commands and parameters are used by computer 36 to provide control signals and information to DAS 32, x-ray controller 28 and gantry motor controller 30. In addition, computer 36 operates a table motor controller 44 which controls a motorized table 46 to position patient 22 in gantries 13 and 14. Specifically, table 46 moves portions of patient 22 through gantry opening 48.

In one embodiment, computer 36 includes a read/write device 50, for example, a floppy disk drive, CD-ROM drive, DVD drive, magnetic optical disk (MOD) device, or any other digital device including a network connecting device such as an Ethernet device for reading instructions and/or data from a computer-readable medium 52, such as a floppy disk, a CD-ROM, a DVD or an other digital source such as a network or the Internet, as well as yet to be developed digital means. In another embodiment, computer 36 executes instructions stored in firmware (not shown). Computer 36 is programmed to perform functions as described herein, and as used herein, the term computer is not limited to integrated circuits referred to in the art as computers, but broadly refers to computers, processors, microcontrollers, microcomputers, programmable logic controllers, application specific integrated circuits, and other programmable circuits, and these terms are used interchangeably herein. Computer 36 can be accessed and controlled by user interface 100. CT/PET system 10 also includes a plurality of PET detectors (not shown) including a plurality of detector elements. The PET detectors and detector array 18 both detect radiation and are both referred to herein as radiation detectors.

An automatic protocol selector 54 is communicatively coupled to DAS 32 and image reconstructor 34 to transmit settings and parameters for use by DAS 32 and image reconstructor 34 during a scan and/or image reconstruction and image review. Although automatic protocol selector 54 is illustrated as a separate component, it should be understood that functions performed by automatic protocol selector 54 may be incorporated into functions performed by, for example computer 36. Accordingly, automatic protocol selector 54 may be embodied in a software code segment executing on a multifunctional processor or may be embodied in a combination of hardware and software.

Control of a system or modality is not limited to a single scan. A user interface may change from a scan state to analysis state seamlessly, and may be able to monitor scan parameters of a scan proceeding, while separately viewing scan results from a prior scan. For instance, a radiologist may elect to monitor a scan proceeding of a torso on one screen, while simultaneously reviewing the results of a head scan for the same or even a different patient.

A CAD processor 55 accepts data from the image reconstructor 34 and performs an analysis of all major organ systems captured in the scan. Prior information, such as lab tests, patient history and prior exams are made available to the CAD processor 55 from the computer 36 to permit a thorough CAD analysis on all available patient data. The CAD analysis automatically identifies each organ and organ system in the scan through analysis of image features/signatures and deformable registration with an anatomical/functional atlas. The atlas contains reference geometry, anatomical and functional ontologies, and structural variance observed in a large patient population. The atlas may represent a large collection of atlases that are formed with age, gender, condition, etc. subpopulations. This would allow the atlas to account for age and other controls in defining the location structure, and variance to be expected in normal and diseased anatomy. The atlas also contains references to the key detection and measurement calculations that can be performed in each body region. These CAD analysis modules are then executed on each body region giving both an overall status of the organ system as well as detailed measurements and findings associated with the organ system.

A CAD analysis module can be constructed to operate on skeletal structures. Shape based operators, such as the 3D Hessian differential geometry operator or the curvature tensor, can be applied throughout the skeletal system to identify low density sheet-like regions that may identify a bone fracture. Shape based operators can also be used to identify bone cancer and metastases as well as other local abnormalities present in bone structure. Another key measurement is the analysis of bone conditions such as osteoporosis, performed on trabecular and cortical bone present globally in the scan and at specific bone locations. These modules will produce findings and measurements which are then transmitted to the computer 36 for display and storage. The findings may also be used by the scanning system to prescribe an additional scan or reconstruction of a local body region with an important finding utilizing any of the available scanning subsystems.

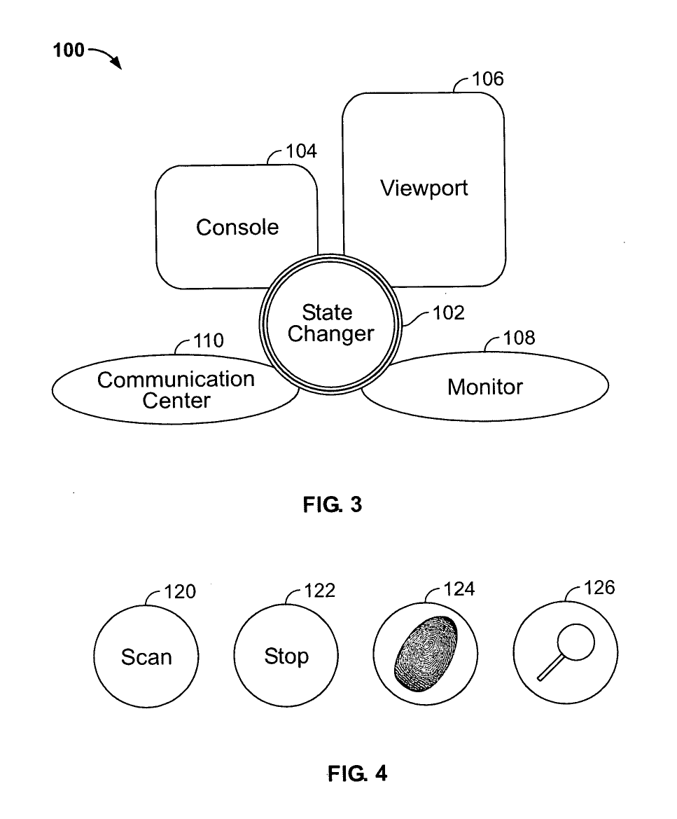

An adaptable user interface 100 is illustrated in FIG. 3. Adaptable user interface 100 may include, but is not limited to, a state changer 102, a console 104, a viewport 106, a monitor 108, and a communication center 110. State changer 102 is a button that allows the user to transform user interface 100 into a different mode of operation. As illustrated in FIG. 4, state changer 102 may be icon driven and may allow a user to initiate a scan 120, stop a scan 122, access the console 124 (i.e. fingerprint access), change to an analysis mode 126, instruct dataflow and save data.

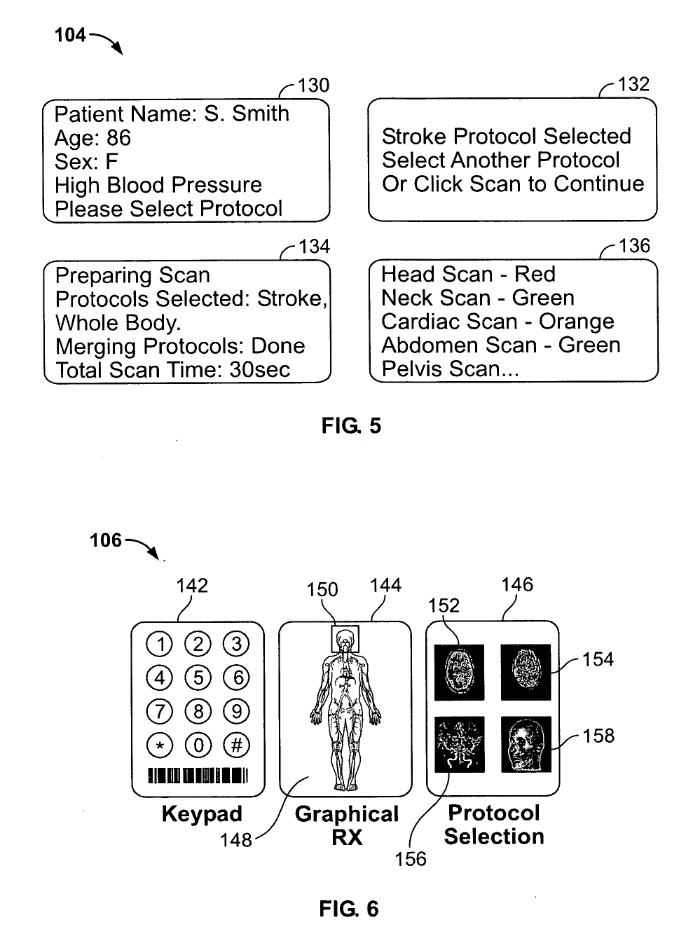

FIG. 5 illustrates examples that console 104 may illustrate if initiated through state changer 102. Console 104 is the main mode of communication between imaging equipment first modality unit 11 or second modality unit 12 and the user. Communication between first modality unit 11 and second modality unit 12 also may include external devices such as a patient database, PACS, HIS/RIS, etc . . . . Imaging systems accessed by user interface 100 need not be mounted back to back and need not be placed in the same hospital suite or even in the same building. System control through user interface 100 is flexible and may be from remote locations and the imaging systems themselves may be located remote from one another as well. Text is displayed to the user in console 104, including but not limited to patient information 130, confirmation of selections 132, current status of workstation scan protocols 134, and current status of the exam 136. Patient information 130 may be entered by a user, or patient information 130 may appear as a result of associating a medical order with a patient record. Current status of the exam 136 may include either current status of the exam or may include analysis of the scan. For example, console 104 may be connected to a diagnostic database (not shown) which automatically analyzes a patient's images from a scan. Based on the analysis, a score of diagnostic relevance is given, in one embodiment, after comparison of imaging data with data from a lookup table, and the diagnostic relevance may be color coded with a menu on the screen to indicate to the user on console 104 the degree of relevance.

Viewport 106 is used to display information for selection by the user during equipment operation and imaging analysis. Input to viewport 106 may be through an input device, such as keyboard 60, mouse 62, or barcode scanner 64. Input to viewport 106 may be through other means as well, such as, but not limited to, voice commands or a touch screen on viewport 106. As illustrated in FIG. 6, keypad 142 may be used on viewport 106 to enter data such as numbers, letters, or symbols. Viewport 106 may also be used to enter a graphical prescription 144 for scanning. Graphical prescription 144 in FIG. 6, for example, illustrates an example of an imaging protocol related to the head area of body 148 as designated and bounded by rectangular indication 150. FIG. 6 also illustrates examples 146, which indicate various examples of different imaging protocols relating to the head area, as designated by marks 152, 154, 156, and 158.

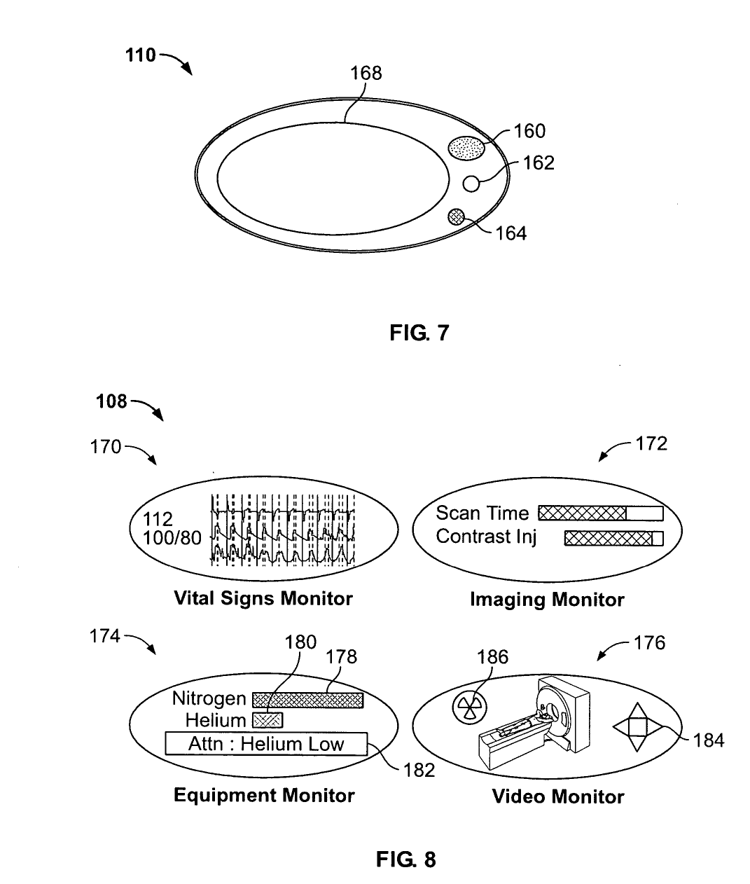

Communication center 110 of user interface 100 enables communication between an operator and a patient, equipment, clinical facilities and staff, an equipment vendor, and/or a service facility. Communication center 110 may also be used to record dictation by a radiologist or other operator during or following an exam. FIG. 7 illustrates an example of a communication center 110. Speaker 160 enables voice communication and enables playing audio transmissions. A flashing light on message indicator 164 indicates a message awaiting the user, which may be accessed if selected and viewed in message area 168. Answer button 162 allows a user to answer calls made to the equipment, such as to external data storage devices, console, etc . . . . The presence of communication center 110 depends on user preference and which state of operation is selected in user interface 100, and communication center 110 is not limited only to the types of interfaces discussed, i.e. operator, patient, and equipment.

Monitor 108, illustrated in FIG. 8, displays information about imaging system 10. A vital signs monitor 170 displays vital signs of a patient, or other patient information (such as family history, genetic disposition, etc . . . ) during a scan. Scan time and other current scan operational parameters may be displayed on an imaging monitor 172. An equipment monitor 174 shows equipment status information, for example a Nitrogen level 178 or a Helium level 180 for a MR system, and monitor 174 may also provide a warning indicator 182 if, for example, helium cryogen level is low. A video monitor 176 may display a patient in imaging system 10. Control 184 may be used to control motion of table 46, for example, during the scan of a patient. Indicator 186 may be used to indicate, for example, radiation danger in the device during utilization of the radiation source.

Allowing system control on user interface 100 enables remote placement of the system control and also allows adjustment of the patient and other scan parameters to occur during a scan. Remote location of the system controls also enables users, operators, radiologists, and others to be remotely removed from imaging system 10, thus decreasing overall radiation exposure. Furthermore, with system controls remotely placed, a skilled operator may be located remotely from the imaging site. Multiple monitors may be displayed at once, enabling a user to monitor patient parameters, scan protocol, the state of operation, and user preference depending on the desire of the user. User interface 100 allows system control over one or a plurality of systems, such as but not limited to, three CT scanners or for instance a MR, CT, and PET scanner. The imaging systems under control of user interface 100 need not be physically located together. For instance, a first imaging system may be used to scan a patient, and the patient may be moved to a second imaging system and scanned using user interface 100.

State changer 102 is a button that allows a user to transform user interface 100 into a different mode of operation. State changer 102 may allow a user to initiate a scan, stop a scan, access the console, or change to analysis mode. User interface 100 will change based on its state of operation. The changes may occur automatically or through user interaction, based on the needs and desires of the user. Example states are as follows:

Inactive state—The system is not currently in operation and no user is logged into the system. Activating user interface 100 may require a thumbprint scan, a name and password, or other means of authenticating a user. In the inactive state, the only user interface 100 required to be visible is state changer 102.

Setup state—This mode is used by a user such as, but not limited to, an imaging technologist, radiologist, or other imaging professional. The user is able to enter patient information and select appropriate scanning protocols 146 through viewport 106. Console 104 will display instructions and information to the user. The user may also elect to view patient vital signs 170, video monitor 176, or other options available to monitor 108 as discussed previously regarding FIG. 8. The user may elect to display communication center 110. During setup, state changer 102 may be used to cancel an imaging session or may be used to change the interface to scan mode to initiate a scan, as discussed previously regarding FIG. 4.

Scan state—This mode is active when a scan is occurring. Imaging monitor 172 is displayed along with console 104, both providing information on scan status. The user may elect to display communication center 110. State changer 102 may be used to stop a scan 122 or switch to analysis mode 126.

Analysis state—This mode is active when reviewing images 126. The mode may be available during the scan itself or following a scan. The user likely to access this mode is the radiologist. Communication center 110 may be active during analysis for the purposes of dictation. Viewport 106 may be used to select parts of the exam to display, change display parameters, zoom in and out, and conduct other viewing options. Console 104 may be displayed to provide the user with instructions 132 or to display other features available on console 104.

Service state—This mode is used by a field engineer or other service personnel. It may be accessed on site or remotely to conduct troubleshooting, servicing, and diagnostic evaluation of imaging system 10. This mode may also be used to monitor equipment 174 during operation for further assistance to service personnel for conducting troubleshooting, servicing, and diagnostic evaluation.

Training state—This mode is used by a technologist or trainer to provide or receive instruction on the use of imaging system 10. Communication center 110 may be used during training sessions to transmit audio from, for instance, an instructor at a remote location to a trainee located on-site, at the location of imaging system 10. Viewport 106 may be used to input data through keypad 142, and view and select protocols 144 and 146. Console 104 may be used during training sessions to display simulated information as discussed above regarding FIG. 5. Monitor 108 may be used during training to simulate patient conditions by displaying, for instance, simulated vital signs monitor 170 or simulated scanning parameters 172.

User interface 100 may be customized based on a number of factors. Based on the needs of the user, and the various responsibilities of different users (i.e. operator, field service engineer, radiologist, instructor, etc . . . ) imaging system 10 through user interface 100 may be customized accordingly, using state changer 102. For instance, user interface 100 may be minimized or monitor 108 may be hidden when imaging system 10 is in an inactive state. Each group has specific requirements and preferences as to how the user interface should work, and certain groups may have access to or may be barred from access to equipment functionality or image analysis. Each group may also desire to scan or analyze data regarding different modalities.

Additionally, the look of user interface may be stored with particular user preferences at each location. Users accessing a system may recall a user interface that is particular for their personal needs. For example, a field engineer and a radiologist, as described above, will access imaging system 10 through user interface 100 and may prefer to use different features provided by user interface 100. By logging in or otherwise accessing the system, the specific user profile can be recalled and displayed for the particular needs of each user.

User interface 100 functionality may be dependent on, and set according to, the particular imaging equipment being used on imaging system 10. For example, pulse sequences would only be accessible on MRI equipment, or X-Ray tube control parameters may be limited to a CT system. A user may be able to set up and limit use to particular modalities and equipment.

User permissions may be controlled by a super-user. For example, an owner of imaging system 10 may desire to limit access to communication center 110 to a radiologist to prevent a non-radiologist from dictating on the system. Additionally, scanning controls may be limited to only users who are licensed professionals.

Functionality of user interface 100 may depend on the physical location of a user. For example, certain locations may be allowed to scan a patient while other locations may be limited to access to communication 110 to transcribe from dictations of a radiologist. Other remote access locations may be limited to, for example, monitor 108, for access to equipment monitor 174.

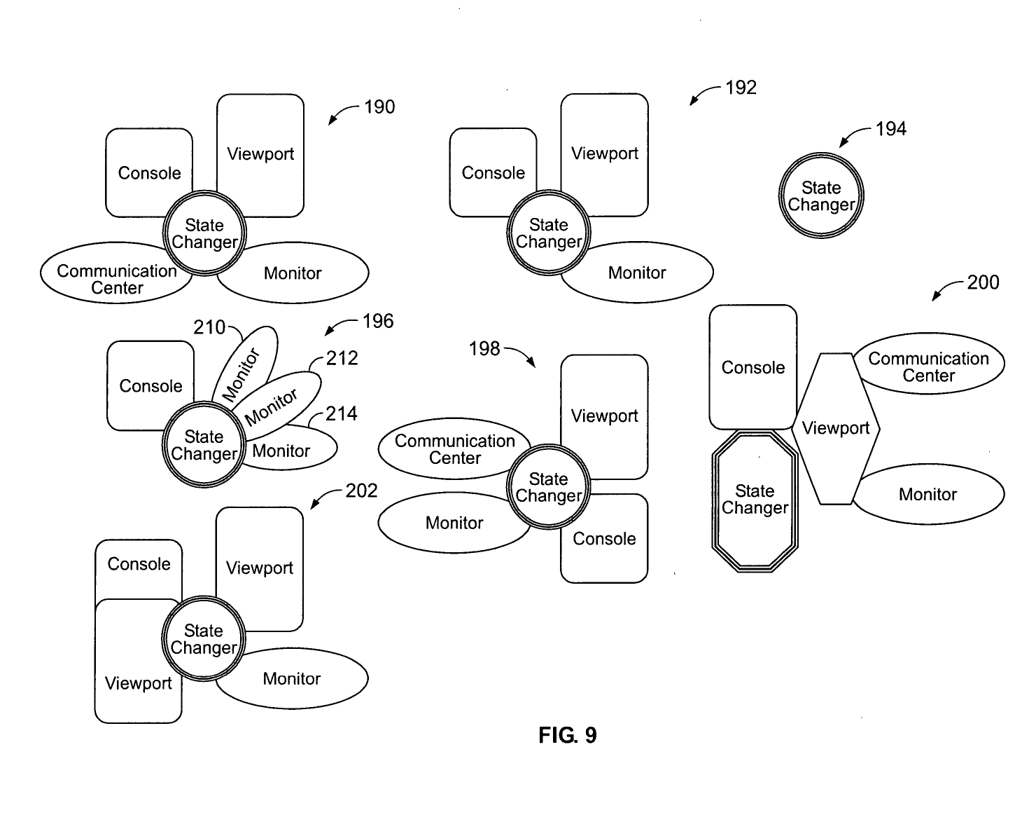

FIG. 9 illustrates examples of user interface configurations. Illustration 190 indicates a standard configuration with a state changer and one each of the four primary functions accessible through state changer 102. Illustration 192 indicates access to a console, viewport, and monitor, but no communication center. Illustration 194 indicates only a state changer, which provides an access point to the user, who may access functionality through state changer 102. Illustration 196 illustrates another user preference, that includes a console and three monitors. Monitors 210, 212, and 214 may, in themselves, each provide separate monitor functions, such as vital signs monitor 170, imaging monitor 172, equipment monitor 174, and video monitor 176. Illustration 198 illustrates the same four functions as shown in illustration 190, but icons are rearranged and re-sized per particular user preferences. Illustration 200, as well, indicates the same four functions as illustration 190, but with icon shapes and locations changed per preferences of the user. Finally, illustration 202 indicates a console, monitor, state changer, and two viewports, all sized and located per preferences of the user and, additionally, the two viewports may have selected to show keypad 142, graphical interface 144, or other features as described and illustrated in FIG. 6.

Additionally, although described in a medical setting, it is contemplated that the embodiments of the invention may be implemented in connection with other imaging systems including industrial CT systems such as, for example, but not limited to, a baggage scanning CT system typically used in a transportation center such as, for example, but not limited to, an airport or a rail station.

During operation, state changer 102 is used to set user preferences as described and illustrated in FIG. 9. State changer 102 is not limited to the examples as illustrated in FIG. 9, but may be used to set up, using state changer 102, any combination of console 104, viewport 106, communication center 110, and monitor 108. A user may set up the combination of functions, icon location, and icon size and shape, according to preferences of the user, and according to the functions on the system that the user has access to.

As analysis becomes more interactive for modern systems, and the speed of scanning becomes faster, state changer 102 enables easy transition from acquisition mode to analysis mode. A suite of interactive displays manages this by allowing the user to select which console is scanner-capable at any time. The display will auto-configure to provide all the interactive data needed to manage acquisition and simplify itself when only display features are desired or required.

The user interface auto-configures to provide the needed data for acquisition. Video surveillance of the patient, respiratory, and cardiac monitoring is integrated into the display. An intercom is provided. A transportable “scan pod” is available to transform any user console into an operator's console. Scan control can be done by moving the pod and changing the state of the state changer 102. The user interface can be reconfigured to meet the needs for all CT users, such as radiologists, scanning technicians, equipment maintenance personnel, and others. In addition, different “pod” configurations can be used to control scanners using modalities other than CT. For instance, a scan pod may be configured to control an MR system, PET system, or other medical imaging system. A single scan pod may be used to control and display multiple scanners of the same or different modality from a single display.

State changer 102 and its embodiments may be an apparatus, a method, a computer, or a program on a computer-readable medium.

State changer 102 may have a designated primary control location or console and others that access the same system would be designated as secondary. This retains control for a super-user that has master control over system functions, who may limit access of the system to other users (such as read-only access), or limited to only certain aspects of the system (such as cryogen levels for a maintenance person). Primary control and secondary control may also be for the purpose of patient safety or operator safety. For instance, a radiologist may be limited so that the radiologist can not control maintenance parameters, leaving system equipment safety to a safety specialist, for instance.

The system may be used for surgical navigation. It may be designed sufficiently flexible such that future surgical developments and procedures may be incorporated and used at a later date. For instance there may be control scheme and icons identified for control of surgical equipment, as well as patient monitoring equipment.

Control consoles may operate independently. For instance, two or more consoles being used by one or more operators at the same or different locations may have separate access to different aspects of the imaging system. Consoles may be located remotely, either in a different hospital suite, a different building, or entirely remote from that location.

The herein described methods and apparatus provide for a single console to control a multiple number of medical systems such as a multiple number of multi-modality systems as well as a multiple number of single modality imaging systems. For example, in one embodiment, a patient in a trauma center is scanned with a CT system and the user can review the CT data while the patient is transported to a MRI system for another scan. The user can then prescribe a MRI scan at the same console used to conduct the CT scan. This saves the user both time and energy than if the user had to move to a different workstation to prescribe the MRI scan.

Additionally, once the CT scan is complete, the user can release the CT (i.e., transfer control to another console), so another user may scan a patient. Note the user also has access to at least one medical database while prescribing the scan, and can use information from the database in prescribing the scan. For example, the database can contain genetic information and the user prescribes the scan accordingly. Additionally, the database can have information specific to the patient and the user uses this patient specific historical or genetic information to prescribe. For example, a patient is brought in for injuries sustained from falling off a skateboard, the user sees that the patient is high risk for a stroke and performs a scan to access brain function, or cerebral blood flow, in addition to a scan for injuries sustained from the fail itself. Accordingly a stroke can be identified as the cause of the fall.

When the analysis determines that certain problems are likely on a percentage of likelihood basis, the potential problems are color coded according to severity as opposed to being color coded based on likelihood. For example, a condition that is small in likelihood but very severe if present is color coded as needing immediate attention or otherwise as very important. The data contained in the database and used for analysis can include physiological data, family history data, patient history data, and correlation data, as well as outcome percentage data that can be global, regional, or facility limited. For example, when the analysis reveals a likely bone fracture in a particular location, the system provides automatically views which facilitate diagnosis of bone fracture in that particular location, as well as treatment options for that type and location of fracture with success rates regionally, globally, and/or facility limited.

Additionally, when the system is operated by a multi-facility organization, the displayed success rate can be the organizations success rate. The system also allows for multiple scan prescriptions for different body portions during a single data acquisition. For example, the user can proscribe a perfusion study for a patient's head and a normal CT scan for the patient's upper body to generate a blended scan.

In one embodiment, the system automatically determines a probability of a problem, and when the probability is greater than a predetermined threshold, the system automatically displays at least one data view associated with that problem. The data view assists the user in diagnosing if the problem exists or not.

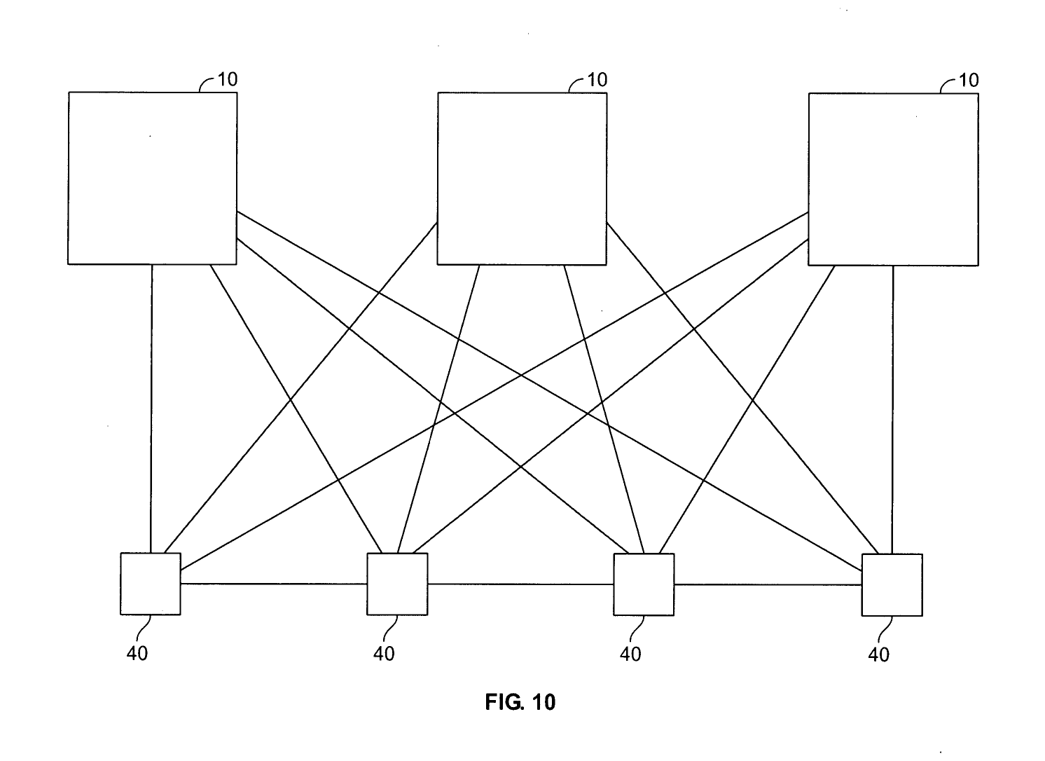

FIG. 10 illustrates a plurality of systems 10 operable by any and all of a plurality of consoles 40. Systems 10 can be of the same modality and/or different modalities or multimodality units.

The above-described state changer and imaging system is a cost-effective and highly reliable means for providing multiple users of an imaging system with separate and unique interfaces to multiple modalities while using a common state changer. It enables users to set up interfaces to an imaging system while enabling a super-user to limit specific functions to individuals, based on their job function and their need to access the imaging system. The herein described methods and systems allow the ability to automatically merge protocols.

The herein described methods and systems also allow for one touch access to specific details via anatomical model (as opposed to basic review and image selection), the ability to automatically perform iterative recon based on comparison findings (i.e. broken hip found, so zoom in on the hip).

A state changer is described above in detail. The configurations set up by the state changer are not limited to the specific embodiments described herein, but rather, functions of each system may be utilized independently and separately and uniquely combined and used by separate users. Configurations described can also be used in combination with other functions accessible through a state changer.

In one embodiment, injector status is one scanning parameter.

While the invention has been described in terms of various specific embodiments, those skilled in the art will recognize that the invention can be practiced with modification within the spirit and scope of the claims.

What is claimed is:

1. A method of operating a user interface comprising:
receiving a first control command at a console operationally coupled to at least a first medical system and a second medical system through a network, wherein the console is in control of the first and second medical systems using the network;
operating the first medical system in accordance with the first control command;
receiving a second control command at the console wherein the second control command includes at least one of a user input and a stored command;
operating the second medical system in accordance with the second control command.

2. A method in accordance with claim 1 further comprising operating the first and second medical systems simultaneously.

3. A method in accordance with claim 1 wherein said operating the medical system comprises selecting from a plurality of states using a state changer, the states indicative of an operation mode of the user interface.

4. A method in accordance with claim 3 wherein a user can operate one of the plurality of states using the first medical system while simultaneously operating a second of the plurality of states using the second medical system.

5. A method of operating a user interface comprising:
receiving at least one first control command using a user interface, wherein the at least one first control command pertains to a first scan of an object with a first imaging device;
performing the received at least one first control command;
receiving at least one second control command using the user interface, wherein the at least one second control command pertains to a second scan of an object with a second imaging device different from the first imaging device; and
performing the at least one second control command.

6. A method in accordance with claim 5 further comprising operating the first and second imaging devices simultaneously.

7. A method in accordance with claim 5 wherein said operating the imaging device comprises selecting from a plurality of states using a state changer, said selecting from a plurality of states further comprising:
selecting an inactive state, wherein the inactive state is indicative of the user interface not being in operation;
selecting a setup state, wherein the user interface includes a plurality of parameters, said selecting a setup state facilitates setting the parameters to facilitate customizing the user interface;
selecting a scan state to facilitate obtaining images from the imaging devices;
selecting an analysis state to facilitate reviewing images from the imaging devices;
selecting a service state to facilitate troubleshooting, servicing, or diagnostic evaluation of the imaging devices; and
selecting a training state to facilitate training a new user of the user interface.

8. A method in accordance with claim 7 wherein a user can operate one of the plurality of states using the first imaging device while simultaneously operating a second of the plurality of states using the second imaging device.

9. A method of operating a user interface comprising:
receiving predetermined thresholds for patient data;
receiving a set of patient data including imaging data of the patient;
comparing the patient data including imaging data to the predetermined thresholds; and
automatically determining a characteristic of the patient from the predetermined thresholds and the patient data including imaging data.

10. A method in accordance with claim 9 wherein automatically determining a characteristic comprises determining a possible diagnosis of the patient using the imaging data and predetermined thresholds.

11. A user interface comprising:
at least two medical systems operatively coupled to a control console, said control console having simultaneous control over said first and second medical systems.

12. A user interface in accordance with claim 11 further comprising a state changer, said state changer allows a user to select from a plurality of states indicative of an operation mode of the user interface.

13. A method in accordance with claim 12 wherein a user can operate one of said plurality of states using said first medical system while simultaneously operating a second of said plurality of states using said second medical system.

14. A user interface in accordance with claim 11 wherein said medical systems are imaging devices and said states further comprises:
an inactive state indicative of the user interface not being in operation;
a setup state to allow a user to select from a plurality of parameters to customize said user interface;
a scan state to obtain images from the imaging devices;
an analysis state to review images from the imaging devices;
a service state to troubleshoot, service, or diagnostically evaluate the imaging devices; and
a training state to facilitate training a new user of the user interface.

15. A method in accordance with claim 14 wherein a user can operate one of said plurality of states using said first imaging device while simultaneously operating a second of said plurality of states using said second imaging device.

16. A computer program embodied on a computer readable medium for operating a user interface, said program comprising at least one code segment that:
receives at least one first control command at a console operationally coupled to at least a first medical system and a second medical system, wherein the console is in control of the first and second medical systems;
operates the first medical system in accordance with the at least one first control command;
receives at least one second control command at the console;
operates the second medical system in accordance with the at least one second control command.

17. A computer program embodied on a computer readable medium in accordance with claim 16, said program further comprising at least one code segment that operates the first and second medical systems simultaneously.

18. A computer program embodied on a computer readable medium in accordance with claim 16, said program further comprising at least one code segment that selects from a plurality of states indicative of an operation mode of the user interface.

19. A computer program embodied on a computer readable medium in accordance with claim 18, said program further comprising at least one code segment that operates one of the plurality of states using the first medical system while simultaneously operating a second of the plurality of states using the second medical system.

20. A computer program embodied on a computer readable medium in accordance with claim 19 wherein the medical systems are imaging devices and said program further comprising at least one code segment that:
selects an inactive state indicative of the user interface not being in operation;
selects a setup state to set parameters to customize the user interface;
selects a scan state to obtain images from the imaging devices;
selects an analysis state to review images from the imaging devices;
selects a service state to troubleshoot, service, or diagnostically evaluate the imaging devices; and
selects a training state to train a new user of the user interface.

21. A method in accordance with claim 9 wherein receiving predetermined thresholds for patient data comprises receiving predetermined thresholds for patient data wherein the thresholds include threshold data relating to a patient physiological data, family history data, patient history data, correlation data, and outcome percentage data limited to at least one of a global population, a regional population, and a facility population.
